(19) United States
(12) Patent Application Publication
(10) Pub. No.: US 2012/0289734 A1
(43) Pub. Date: Nov. 15, 2012

(54) ALTERNATIVE SYNTHESIS OF 1,1-SUBSTITUTED OLEFINS HAVING ELECTRON-WITHDRAWING SUBSTITUENTS

(75) Inventors: Carsten Friese, Duesseldorf (DE); Dieter Kaufmann, Goslar (DE); Vasu Govardhana Reddy Peddiahgari, Andhra Pradesh (IN)

(73) Assignee: Henkel AG & Co. KGaA, Duesseldorf (DE)

(21) Appl. No.: 13/458,664

(22) Filed: Apr. 27, 2012

Related U.S. Application Data
(63) Continuation of application No. PCT/EP2010/059153, filed on Jun. 28, 2010.

(30) Foreign Application Priority Data
Oct. 29, 2009 (DE) 10 2009 046 131.0

(51) Int. Cl. C07C 253/30; C07C 67/465

(52) U.S. Cl. 558/427; 560/8; 560/80

(57) ABSTRACT

A three-stage method for synthesizing 1,1-disubstituted olefins is provided.

The subject matter of the invention is a process for manufacturing 1,1-disubstituted olefins.

The carbon-carbon double bond is one of the most important functional groups of monomeric compounds that are used in the field of adhesive technology. For example it forms the basis and the characteristic feature of so-called polymerization adhesives. Monomers that comprise a carbon-carbon double bond are, however, also the basis for a series of additional classes of adhesives such as for example for dispersion adhesives or hot melt adhesives.

The cyanoacrylate adhesives occupy a prominent position within the class of the polymerization adhesives. In cyanoacrylate esters the charge equilibrium is shifted by the cyano and ester groups present on one carbon atom, such that the possibility exists for the addition of nucleophilic atom groupings, for example OH⁻ ions. There results an activated adduct that initiates an ionic chain polymerization. The extent of the charge transfer and thereby the rate of formation of the activated adduct is decisively influenced by the ester group that is present. Consequently, differentiated curing rates of the cyanoacrylate adhesives can be realized by the choice of the alcohols used for the esterification. As the described mechanism proceeds at a high rate and an adequate initial strength for further processing is already attained after some seconds, cyanoacrylates are marketed as so-called instant adhesives.

The above example illustrates the enormous significance of 1,1-substituted olefins in the field of adhesives and simultaneously refers to the role of the substitution pattern for obtaining different property profiles of the corresponding adhesives. Against this background, syntheses for the preparation of 1,1-substituted olefins—in particular with electron withdrawing substituents—play a key role in the further development of adhesives and their manufacturing processes.

An enantioselective and diastereoselective synthesis of alpha-chloroacrylonitrile is described for example by Wang, Liu and Deng (J. Am. Chem. Soc. 2006, 128, 3928). The process proceeds as a tandem addition-protonation reaction of trisubstituted carbon nucleophiles in the presence of 6″-OH-cinchona alkaloids as the catalyst.

There is a continued need for synthetic processes for the preparation of 1,1-disubstituted olefins which offer, in as few steps as possible, direct access to monomeric, geminal disubstituted olefins, in particular with electron withdrawing substituents. Accordingly, the object of the present invention is to provide a process that with inexpensive and widely available starting materials makes possible a synthesis in few steps of 1,1-disubstituted olefins. In particular, an easily carried out method for the introduction of additional electron withdrawing substituents into electron-poor olefins should be provided.

The object is achieved by a process based on a three step synthesis, consisting of a cycloaddition, a substitution and an elimination sequence. Accordingly, the subject matter of the invention is a process for the synthesis of 1,1-disubstituted olefins which in a first step includes the reaction of a 1,3-diene with an olefin of the general Formula (I),

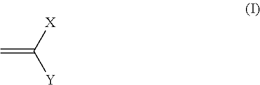

in which X is an electron withdrawing substituent and Y is a hydrogen atom or a halide atom, wherein X and Y are not identical,
to an unsaturated cyclic compound comprising the substituents X and Y;
in a second step the substitution of Y by a substituent W, selected from a C₁-C₁₂ hydrocarbon group, a C₂-C₁₂ carboxylic acid ester group, a C₃-C₁₂ alkoxy carbonylalkyl group, a C₂-C₁₂ alkanoyl, C₄-C₁₂ cycloalkanecarbonyl or C₆-C₁₂ (hetero)arenecarbonyl group, a C₁-C₁₂ hydroxyalkyl group, a C₂-C₁₂ phosphonic acid ester group, a C₁-C_{12 sulfonyl group, a C}₁-C₁₂ sulfinyl group, a C₁-C₁₂ sulfonic acid ester group, an aldehyde group, a carbamoyl group, a halide atom and a cyano group; and
in a third step the elimination of a 1,1-disubstituted olefin of the general Formula (II),

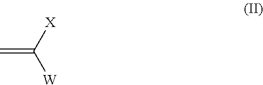

in which X and W have the above meaning.

A 1,3-diene is understood to mean an optionally substituted aliphatic, cycloaliphatic or even aromatic hydrocarbon which possesses two conjugated double bonds that are linked together with a single bond. Generally, they concern 1,3-dienes that can be reacted with suitable dienophiles in the context of a Diels-Alder reaction. In the case of supposedly aromatic compounds, the assumption that follows the general understanding of the person skilled in the art applies that at least in the reactive area of the molecule, localized double bonds are present rather than a delocalized aromatic bond system. Exemplary suitable 1,3-dienes are butadiene, cyclopentadiene, furan and anthracene.

An aromatic compound that in the context of a Diels-Alder reaction can react with suitable dienophiles is particularly preferred as the 1,3-diene, especially anthracene.

An electron withdrawing group is understood to mean a substituent that by a mesomeric and/or inductive effect causes a shift in the electron density from the remainder of the molecule towards itself and thereby reduces the electron density of the remainder of the molecule, namely to a greater extent than would be caused by a hydrogen atom located at the same position. Known electron withdrawing substituents are for example the nitro group, aliphatic and aromatic acyl groups, the aldehyde group, sulfonyl groups, the trifluoromethyl group, carboxylic acid ester groups, the cyano group and halide atoms such as chlorine and fluorine.

The substituent X preferably comprises at least one carbon atom and at least one heteroatom, selected from nitrogen (N), sulfur (S) and oxygen (O). X is particularly preferably a cyano (—CN) or a carboxylic acid ester group —COOR, in which R is an alkyl group. In particular, R is a methyl, ethyl, n-propyl, iso-propyl, n-butyl or iso-butyl group and quite particularly preferably a methyl or ethyl group.

The substituent Y is preferably a hydrogen atom.

An unsaturated compound is understood to many any compound that possesses at least one carbon-carbon double bond or triple bond. A cyclic compound is understood to many any compound, in which some or all non-hydrogen atoms are arranged in ring structures. The unsaturated, cyclic compound comprising the substituents X and Y obtained as the result of the first process step is preferably a bicyclic or polycyclic compound. These are understood to mean a compound that comprises two (bicyclic) or more (polycyclic) ring structures that can be differentiated from each other, wherein the ring structures optionally differ from each other only in some of the structural atoms.

The reactions in the first step of the process according to the invention are preferably carried out under reflux conditions in aromatic solvents such as toluene, benzene or xylene, wherein ortho-xylene is particularly preferred as the solvent. The reaction times can in principle be freely determined. The reactions are preferably carried out for at least 5, particularly preferably for at least 15 and quite particularly preferably for at least 18 hours under reflux conditions.

In the first step of the process according to the invention, the stoichiometric ratio or the mole ratio (mol/mol) of the olefin of the general Formula (I) to the 1,3-diene is preferably at least 3:1. Such an excess of the olefin of Formula (I) enables good yields, which generally range from 50 to 70% in the first step of the process.

The second step of the process according to the invention realizes the substitution of Y by a substituent W, selected from a C₁-C₁₂ hydrocarbon group, a C₂-C₁₂ carboxylic acid ester group, a C₃-C₁₂ alkoxy carbonylalkyl group, a C₂-C₁₂ alkanoyl, C₄-C₁₂ cycloalkanecarbonyl or C₆-C₁₂ (hetero)arenecarbonyl group, a C₁-C₁₂ hydroxyalkyl group, a C₂-C₁₂ phosphonic acid ester group, a C₁-C₁₂ sulfonyl group, a sulfinyl group, a C₁-C₁₂ sulfonic acid ester group, an aldehyde group, a carbamoyl group, a halide atom and a cyano group.

A C₁-C₁₂ hydrocarbon group is understood to mean an organic group that consists only of carbon and hydrogen atoms and comprises 1 to 12, preferably 1, 2, 3, 6 or 7 carbon atoms. Therefore it can be both an aliphatic as well as an alicyclic or an aromatic group.

A C₂-C₁₂ carboxylic acid ester group is understood to mean a group of the general Formula —C(O)OR that comprises 2 to 12, preferably 2, 3 or 4 carbon atoms, wherein R stands for an unsubstituted or substituted alkyl or aryl group. The term alkyl includes here and also in the following definitions, in so far as is not more clearly defined, principally both aliphatic as well as alicyclic alkyl groups.

A C₃-C₁₂ alkoxycarbonyl alkyl group is understood to mean a group of the general Formula —R¹C(O)OR² that comprises 3 to 12, preferably 3, 4 5 carbon atoms, wherein R¹ stands for alkylene group and R² for an unsubstituted or substituted alkyl or aryl group.

A C₂-C₁₂ alkanoyl group is understood to mean a group of the general Formula —C(O)R that comprises 2 to 12, preferably 2, 3 or 4 carbon atoms, wherein R stands for an unsubstituted or substituted aliphatic alkyl group.

A C₄-C₁₂ cycloalkanecarbonyl group is understood to mean a group of the general Formula —C(O)R that comprises 4 to 12, in particular 5, 6, 7 or 8 carbon atoms, wherein R stands for an unsubstituted or substituted alicyclic group.

A C₆-C₁₂ (hetero)arenecarbonyl group is understood to mean a group of the general Formula —C(O)R that comprises 6 to 12, preferably 5, 6, 7 or 8 carbon atoms, wherein R stands for an aromatic or heteroaromatic unsubstituted or substituted group.

A C₁-C₁₂ hydroxyalkyl group is understood to mean a group of the general Formula —R(OH)_{R} that comprises 1 to 12, preferably 1, 2 or 3 carbon atoms, wherein R stands for an optionally not otherwise substituted or unsubstituted alkyl group and x for a whole number from 1 to 4 and preferably for 1.

A C₂-C₁₂ phosphonic acid ester group is understood to mean a group of the general Formula —P(O)(OR)₂ that comprises 2 to 12, preferably 2, 3 or 4 carbon atoms, wherein R stands for the same or different, unsubstituted or substituted alkyl or aryl groups.

A C₁-C₁₂ sulfonyl group is understood to mean a group of the general Formula —S(O)₂R that comprises 1 to 12, preferably 1, 2, 3, 6 or 7 carbon atoms, wherein R stands for an unsubstituted or substituted alkyl or aryl group, in particular for an alkyl group.

A C₁-C₁₂ sulfinyl group is understood to mean a group of the general Formula —S(O)R that comprises 1 to 12, preferably 1, 2, 3, 6 or 7 carbon atoms, wherein R stands for an unsubstituted or substituted alkyl or aryl group, in particular for an alkyl group.

A C₁-C₁₂ sulfonic acid ester group is understood to mean a group of the general Formula —S(O)₂OR that comprises 1 to 12, preferably 1, 2, 3, 6 or 7 carbon atoms, wherein R stands for an unsubstituted or substituted alkyl or aryl group, in particular for an alkyl group.

The aldehyde group, the carbamoyl group and the cyano group are understood to mean the —CHO group, the —C(O)NH₂ group and the —CN group respectively.

In the process according to the invention, the substituent W is preferably selected from a cyano group as well as from a carboxylic acid ester group, a phosphonic acid ester group, a sulfonyl group, a sulfinyl group and a sulfonic acid ester group. W is particularly preferably a carboxylic acid ester group, preferably comprising 2, 3 or 4 carbon atoms. Carboxylic acid ester groups can be introduced with comparatively high yields of between 70 and 90%. X and W are quite particularly preferably a cyano group and a carboxylic acid ester group respectively.

In the second step of the process according to the invention, the stoichiometric ratio of the compound capable of contributing the substituent W to the unsaturated cyclic compound that comprises the substituents X and Y preferably has values of 1:1 to 1.3:1, in particular 1:1 to 1.2:1. A slight excess of the substituent to be incorporated advantageously shifts the reaction rate in favor of the substitution product. Furthermore, at the end of the reaction this slight excess of the compound capable of contributing the substituent W can be relatively easily removed, for example by chromatography.

In a specific embodiment of the process according to the invention, Y is a hydrogen atom, and the substitution of Y by W—the second step of the process according to the invention—includes at least two partial steps: a first partial step, in which a reactive species is formed from the reaction of a base with the unsaturated cyclic compound that comprises the substituents X and Y, and a second partial step, in which a compound capable of contributing the substituent W is added to the reactive species.

As the base for the first partial step, in principle any base can be used whose basicity is sufficient to deprotonate the carbon atom that is acidified by the electron attraction of the substituent X. The base that is used in the first partial step preferably comprises a nitrogen atom. The base is particularly preferably an alkali metal salt, quite particularly preferably a lithium salt, of a secondary amine. In particular, the base that is used in the first partial step is lithium diisopropylamide. The reactive species that is produced by the base consequently has anionic character and can, as has been shown, be reacted with a series of electrophiles.

The substitution of the hydrogen atom Y by a substituent W is preferably carried out at temperatures below 0° C., particularly preferably below −30° C., quite particularly preferably below −50° C. and especially below −65° C. This means that both partial steps of the above described specific embodiment of the process according to the invention are preferably carried out at a temperature below 0° C., particularly preferably below −30° C., quite particularly preferably below −50° C. and especially below −65° C.

Preferred solvents for the above described specific embodiment of the process according to the invention are cyclic ethers, especially tetrahydrofuran. The preferred reaction times for the first and the second partial steps are each at least 10 minutes, particularly preferably at least 20 minutes and especially at least 25 minutes.

The compound capable of contributing the substituent W in the second partial step of the above described embodiment is preferably characterized in that it is capable of releasing W as the electrophile, thereby inserting these substituents into the formed reactive species. Exemplary suitable reagents are:

dialkyl sulfates for introducing alkyl groups, especially dimethyl sulfate and diethyl sulfate; chlorocarbonic acid esters for introducing carbonic acid alkyl ester groups or carbonic acid aryl ester groups, especially chlorocarbonic acid methyl ester, chlorocarbonic acid ethyl ester as well as chlorocarbonic acid isobutyl ester; sulfonyl chlorides, with which surprisingly a chlorine atom can be introduced, especially methanesulfonyl chloride and p-tolylsulfonyl chloride (tosyl chloride); sulfonic acid anhydrides for introducing sulfonyl groups, especially trifluoromethanesulfonic acid anhydride; phosphodialkyl ester chlorides (phosphoric acid dialkyl ester chlorides), phosphodiaryl ester chlorides or phosphoric acid triesters for introducing phosphonic acid ester groups, especially phosphodimethyl ester chloride and phosphodiphenyl ester chloride; also organochlorosilanes such as for example trimethylchlorosilane, with which triorganosilyl groups such as for example trimethylsilyl groups can be introduced; as well as allyl and benzyl halides, especially the corresponding chlorides, for introducing allyl or benzyl groups. Moreover, p-tolylsulfonyl cyanide for example can also be used to introduce cyano groups.

The third step of the process according to the invention includes the cleavage of a 1,1-disubstituted olefin of the general Formula (II) from the X and W substituted, unsaturated, cyclic compound formed in the first and second steps. The cleavage of the 1,1-disubstituted olefin of the general Formula (II) is preferably carried out under the action of heat or by electromagnet induction.

The cleavage of a 1,1-disubstituted olefin of the general Formula (II) is preferably carried out by means of electromagnetic induction, in that an X and W substituted, unsaturated, cyclic compound is brought into contact with an electromagnetically induction heatable solid heating medium that is located inside a reactor,

and is heated by electromagnetic induction with the help of an inductor, wherein the 1,1-disubstituted olefin of the general Formula (II) is formed from the X and W substituted, unsaturated, cyclic compound. The 1,1-disubstituted olefin of the general Formula (II) can then be separated from the solid heating medium.

The heating medium consists of an electrically conductive material that is heated by the action of an alternating electrical field. It is preferably selected from materials that possess a very high surface to volume ratio. For example the heating medium can be selected in each case from electrically conductive filings, wires, meshes, wool, membranes, porous frits, pipe bundles (of three or more pipes), rolled up metal foils, foams, packing materials such as for example granules or pellets, Raschig rings and particularly particles that preferably have an average diameter of not more than 1 mm. For example, mixed metallic elements can be employed as the heating medium, as are used for static mixers. In order to be heatable by electromagnetic induction, the heating medium is electrically conductive, for example metallic (wherein it can be diamagnetic) or it exhibits enhanced interaction towards diamagnetism with a magnetic field and in particular is ferromagnetic, ferrimagnetic, paramagnetic or super-paramagnetic. In this regard it is immaterial whether the heating medium is of an organic or inorganic nature or whether it contains both inorganic as well as organic components.

In a preferred embodiment, the heating medium is selected from particles of electrically conductive and/or magnetizable solids, wherein the mean particle size of the particles is from 1 to 1000, especially from 10 to 500 nm. The mean particle size and when necessary also the particle size distribution can be determined for example by light scattering. Magnetic particles are preferably selected, for example ferromagnetic or super-paramagnetic particles, which exhibit the lowest possible remanence or residual magnetism. This has the advantage that the particles do not adhere to each other. The magnetic particles can be in the form of “ferrofluids” for example, i.e. liquids, in which nanoscale ferromagnetic particles are dispersed. The liquid phase of the ferrofluid can then serve as the reaction medium.

Magnetizable particles, in particular ferromagnetic particles, which exhibit the desired properties, are known from the prior art and are commercially available.

The commercially available ferrofluids may be cited as an example. Examples for the manufacture of magnetic nano-particles, which can be used in the context of the process according to the invention, can be found in the article by Lu, Salabas and Schüth: “Magnetische nano-Partikel: Synthese, Stabilisierung, Funktionalisierung and Anwendung”, Angew. Chem. 2007, 119, pp. 1242 to 1266.

Suitable nano-particles with different compositions and phases are known. The following examples may be cited: pure metals such as Fe, Co and Ni, oxides such as Fe₃O₄ and gamma-Fe₂O₃, spinel type ferromagnets such as MgFe₂O₄, MnFe₂O₄ and CoFe₂O₄ as well as alloys such as CoPt₃ and FePt. The magnetic nano-particles can be of a homogeneous structure or can possess a core-shell structure. In the latter case the core and shell can consist of different ferromagnetic or also antiferromagnetic materials. However, embodiments are also possible, in which a magnetizable core that can be for example ferromagnetic, antiferromagnetic, paramagnetic or super-paramagnetic, is surrounded by a non-magnetic material. An organic polymer for example, can represent this material. Or the shell consists of an inorganic material such as for example silica or SiO₂. A coating of this type can prevent a chemical interaction between the reaction medium or the reactants with the material of the magnetic particle itself. In addition, the shell material can be surface modified, without the material of the magnetizable core interacting with the functionalizing entity. In this regard, a plurality of particles of the core material can be enclosed together into a shell of this type.

Nano-scale particles of superparamagnetic substances for example can be employed as the heating medium and are selected from aluminum, cobalt, iron, nickel or their alloys, metal oxides of the type n-maghemite (gamma-Fe₂O₃), n-magnetite (Fe₃O₄) or ferrites of the type MeFe₂O₄, wherein Me is a divalent metal selected from manganese, copper, zinc, cobalt, nickel, magnesium, calcium or cadmium.

Preferably the mean particle size of these particles is ≧100 nm, preferably ≧51 nm and particularly preferably ≧30 nm.

An exemplary suitable material is available from Evonik (formally Degussa) under the name MagSilica^{R}. In this material, iron oxide particles having a size between 5 and 30 nm are embedded in an amorphous silica matrix. Such iron oxide-silicon dioxide composite particles, which are described in more detail in the German patent application DE 101 40 089, are particularly suitable.

These particles can comprise super-paramagnetic iron oxide domains with a diameter of 3 to 20 nm. This is understood to mean super-paramagnetic regions that are spatially separated from one another. The iron oxide can be present in these domains in a single modification or in various modifications. A particularly preferred super-paramagnetic iron oxide domain is gamma-Fe₂O₃, Fe₃O₄ and mixtures thereof.

The content of the super-paramagnetic iron oxide domains of these particles can be between 1 and 99.6 wt %. The individual domains are separated from one another and/or surrounded by a non-magnetizable silicon dioxide matrix. The region containing a content of the super-paramagnetic iron oxide domains is preferably >30 wt %, particularly preferably >50 wt %. The achievable magnetic effect of the inventive particle also increases with the content of the super-paramagnetic regions. The silicon dioxide matrix also stabilizes the oxidation level of the domain in addition to the spatial separation of the super-paramagnetic iron oxide domains. Thus, for example, magnetite is stabilized as the superparamagnetic iron oxide phase by a silicon dioxide matrix. These and further properties of these particles that are particularly suitable for the present invention are listed in more detail in DE 101 40 089 and in WO 03/042315.

Furthermore, nano-scale ferrites such as those known for example from WO 03/054102 can be employed as the heating medium. These ferrites possess the composition (M^{a}_{1-X-y} M^{b}ₓ Fe″_{y}) Fe′″₂O₄, in which

M^{a} is selected from Mn, Co, Ni, Mg, Ca, Cu, Zn, Y and V,
M^{b} is selected from Zn and Cd,
x stands for 0.05 to 0.95, preferably 0.01 to 0.8,
y stands for 0 to 0.95 and
the sum of x and y is maximum 1.

The particles that can be heated by electromagnetic induction can represent the heating medium without any additional additives. However, it is also possible to mix the particles that can be heated by electromagnetic induction with other particles that cannot be heated by electromagnetic induction. Sand for example can be used. Accordingly, the inductively heatable particles can be diluted with non-inductively heatable particles. This allows an improved temperature control. In another embodiment, the inductively heatable particles can be admixed with non-inductively heatable particles that have catalytic properties for the chemical reaction to be carried out or that participate in other ways in the chemical reaction. These particles are then not directly heated by electromagnetic induction, but rather indirectly, in that they are heated by contact with the heatable particles or by heat transfer from the reaction medium.

If nano-scale electromagnetically inductively heatable particles are blended with coarser non-inductively heatable particles, then this can lead to a decreased packing density of the heating medium.

It goes without saying that the nature of the heating medium and the design of the inductor have to be matched to each other in such a way that permits the reaction medium to be heated up as required. A critical variable for this is firstly the rated power of the inductor in watts as well as the frequency of the alternating field generated by the inductor. In principle, the greater the mass of the heating medium to be inductively heated, the higher will be the chosen power. In practice, the achievable power is limited primarily by the ability to cool the generator required for supplying the inductor.

Particularly suitable inductors generate an alternating field with a frequency in the range of about 1 to about 100 kHz, preferably from 10 to 80 kHz and particularly preferably from about 10 to about 30 kHz. Inductors of this type together with the associated generators are commercially available, for example from IFF GmbH in Ismaning (Germany).

Thus the inductive heating is preferably carried out with an alternating field in the medium frequency range. This has the advantage, when compared with an excitation with higher frequencies, for example with those in the high frequency range (frequencies above 0.5, especially above 1 MHz), that the energy input into the heating medium can be better controlled.

In the case of the cleavage of the 1,1-disubstituted olefin of the general Formula (II) by means of electromagnetic induction, reactors are preferably used that are made of a material that neither screens nor absorbs the electromagnetic alternating field produced by the inductor and is therefore itself not heated up. Consequently, metals are unsuitable. For example it can consist of plastic, glass or ceramics (such as for example silicon carbide or silicon nitride). The latter is particularly suitable for reactions at high temperatures and/or under pressure.

In principle all embodiments, tolerance intervals, ingredients and other features of the process according to the invention that are described in the context of the present document can be realized in all possible and non-mutually exclusive combinations. Combinations of features attributed as preferred are themselves likewise inventively preferred.

### EXAMPLES

### Step 1

### Cycloadduct of Anthracene and Ethyl Acrylate (3)

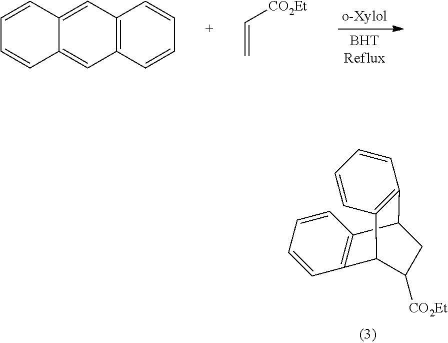

A mixture of anthracene (4.0 g, 22 mmol), ethyl acrylate (12 ml, 110 mmol) and 2,6-di-tert-butyl-4-methylphenol (BHT) (50 mg) in o-xylene (30 ml) was stirred in a 250 ml round-bottomed flask for 24 hours under reflux. The reaction mixture was then cooled, unreacted anthracene was filtered off and the solvent was removed under reduced pressure. The crude product was recrystallized from hexane. The product (3) was obtained in 70% yield. The NMR spectral data were in agreement with the literature values (Chung, Y; Duerr, B. F.; McKelvy, T. A.; Nanjappan, P.; Czarnik, A. W. J. Org. Chem. 1989, 54, 1018-1032).

### Cycloadduct of Anthracene and Acrylonitrile (5)

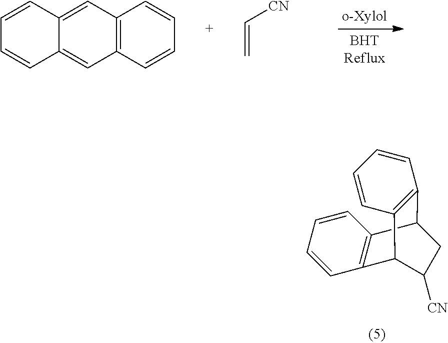

The compound (5) was produced analogously to the synthetic process for compound (3). The crude product was recrystallized from ether. The product (5) was obtained in 70% yield. The NMR spectral data were in agreement with the literature values (e.g. Brown, P.; Cookson, R. C; Tetrahedron, 1965, 21, 1993-1998).

### Cycloadduct of Anthracene and 2-Chloroacrylonitrile (7)

The compound (7) was produced analogously to the synthetic process for compound (3). The crude product was purified by flash-column chromatography on silica gel (10% ethyl acetate/pentane). The compound (7) was obtained in 50% yield.

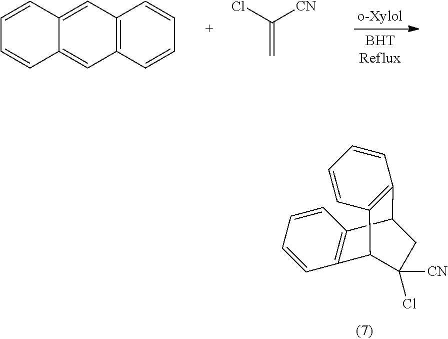

¹H-NMR (CDCl₃, 200 MHz) δ=7.47-7.16 (m, 8H, Ar—H), 4.71 (s, 1H), 4.38, 4.35 (dd, 1H, J=2.2, 2.4 Hz), 2.85, 2.78 (dd, 1H, J=2.6, 2.6 Hz), 2.38, 2.31 (dd, 1H, J=2.4, 2.6 Hz).

¹³C-NMR (CDCl₃, 50 MHz) δ=141.77, 141.07, 137.44, 136.87, 128.03, 127.69, 127.12, 127.00, 126.55, 126.16, 123.77, 123.57, 119.64, 55.79, 55.51, 46.92, 43.30.

### Step 2

Reaction of the Cycloadduct (5) with Dimethyl Sulfate (8)

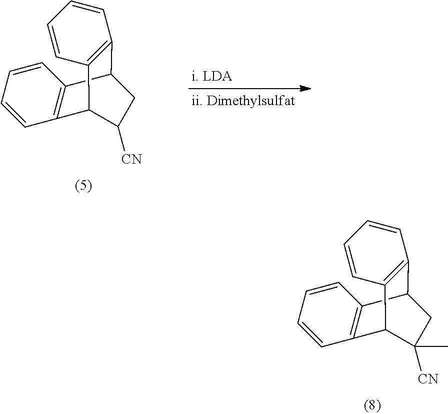

To a solution of the cycloadduct (5) (1.151 g, 5.0 mmol) in THF at −78° C. was added using a syringe a 2 M solution of LDA in THF (3 ml). After 30 minutes a solution of dimethyl sulfate (0.52 ml, 5.5 mmol) in THF was added drop wise at −78° C. After a further 20 minutes water (20 ml) was added and the reaction mixture was warmed to room temperature. The aqueous phase was extracted with ethyl acetate (3×30 ml). The assembled organic phases were washed with NaCl solution (50 ml), dried over sodium sulfate, filtered and concentrated under reduced pressure, whereupon a yellow oil was obtained. The product (8) was purified by flash column chromatography (silica gel, 4% ethyl acetate/pentane) and obtained as a white solid (0.8 g, 65%).

¹H-NMR (CDCl₃, 400 MHz) δ=7.43-7.12 (m, 8H, Ar—H), 4.30 (s, 1H), 4.25 (s, 1H), 2.36, 2.33 (dd. 1H, J=2.4, 2.5 Hz), 1.65, 1.62 (dd, 1H, J=2.4, 2.6 Hz), 1.19 (s, 3H).

¹³C-NMR (CDCl₃, 100 MHz) δ=142.23, 141.94, 140.50, 138.48, 127.12, 127.04, 126.53, 126.22, 125.37, 123.71, 123.57, 53.22, 43.65, 42.19, 36.56, 26.82.

Reaction of the Cycloadduct (3) with Dimethyl Sulfate (9)

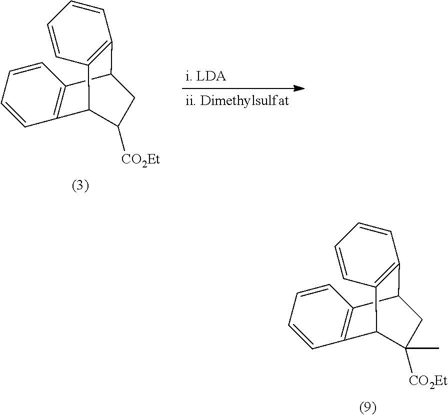

The compound (9) was produced analogously to the synthetic process for compound (8). The crude product was purified by flash-column chromatography on silica gel (3% ethyl acetate/pentane). The compound (9) was obtained in 63% yield.

¹H-NMR (CDCl₃, 400 MHz) δ=7.38-7.10 (m, 8H, Ar—H), 4.37 (s, 1H), 4.32 (s, 1H), 4.04-4.02 (q, 2H), 2.80, 2.77 (dd, 1H, J=2.6, 2.5 Hz), 1.47, 1.44 (dd, 1H, J=2.7, 2.4 Hz), 1.24 (t, 3H), 1.12 (s, 3H).

¹³C-NMR (CDCl₃, 100 MHz) δ=176.65, 143.83, 143.17, 141.17, 140.57, 126.28, 126.05, 125.48, 124.81, 123.49, 123.10, 60.78, 52.82, 48.46, 44.43, 38.73, 26.73, 14.23.

Reaction of the Cycloadduct (5) with Ethyl Chloroformate (10b)

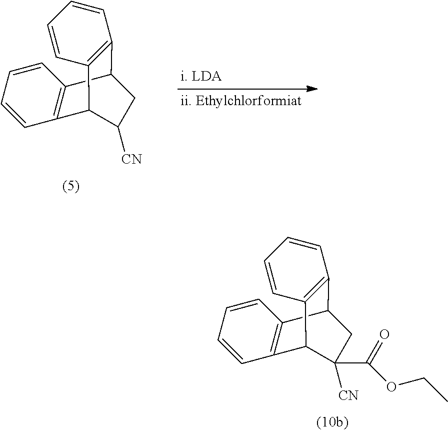

The compound (10b) was produced analogously to the synthetic process for compound (8). The crude product was purified by flash-column chromatography on silica gel (4% ethyl acetate/pentane). The compound (10b) was obtained in 86% yield.

¹H-NMR (CDCl₃, 400 MHz) δ=7.54-7.14 (m, 8H, Ar—H), 4.92 (s, 1H), 4.48 (t, 1H), 4.24-4.16 (m, 2H), 2.87, 2.84 (dd. 1H, J=3.1, 2.7 Hz), 2.27, 2.23 (dd, 1H, J=2.6, 2.6 Hz), 1.32 (t, 3H).

¹³C-NMR (CDCl₃, 100 MHz) δ=166.79, 143.00, 142.36, 137.98, 137.12, 127.61, 127.55, 126.64, 126.36, 125.84, 125.06, 123.91, 123.59, 119.88, 63.24, 51.78, 47.32, 43.18, 37.96, 14.10.

Reaction of the Cycloadduct (3) with Ethyl Chloroformate (11)

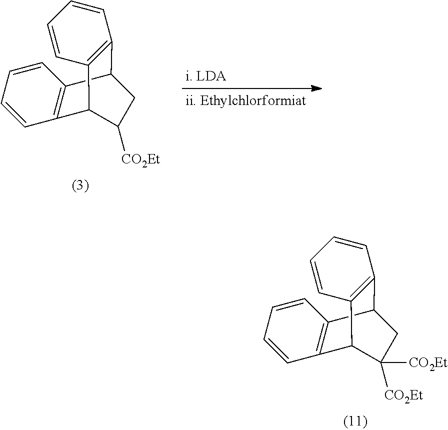

The compound (II) was produced analogously to the synthetic process for compound (8). The crude product was purified by flash-column chromatography on silica gel (6% ethyl acetate/pentane). The compound (II) was obtained in 72% yield.

¹H-NMR (CDCl₃, 400 MHz) δ=7.34-7.08 (m, 8H, Ar—H), 4.98 (s, 1H), 4.34 (s, 1H), 4.25-3.97 (m, 4H), 2.49 (d, 2H, 2.6 Hz), 1.19 (t, 6H).

¹³C-NMR (CDCl₃, 100 MHz) δ=170.25, 143.99, 139.80, 126.44, 125.71, 125.69, 123.30, 61.69, 59.73, 49.63, 43.89, 36.41, 14.05.

Reaction of the Cycloadduct (5) with Methylsulfonyl Chloride (7)

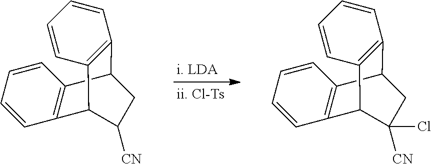

The compound (7) was produced analogously to the synthetic process for compound (8). The crude product was purified by flash-column chromatography on silica gel (4%) ethyl acetate/pentane). The compound (7) was obtained in 60% yield. The NMR spectral data were in overall agreement with the literature values.

### Step 3

### Thermolysis of the Compound (8)

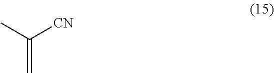

In a bulb tube distillation apparatus, compound (8) (0.490 g, 2 mmol) was heated to 180° C. in a 10 ml flask and distilled. Compound 15 (100 mg) was collected as an oil.

¹H-NMR (Benzene-d₆, 200 MHz) δ=5.11, 5.09 (dd, 1H, 1.1, 0.7 Hz), 4.77, 4.76 (dd, 1H, 1.1, 0.7 Hz), 1.23 (t, 3H).

¹³C-NMR (Benzene-d₆, 50 MHz) δ=130.08, 118.95, 118.29, 20.03.

### Thermolysis of the Compound (10b)

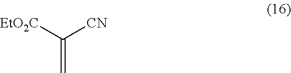

Compound (16) was obtained as an oil analogously to the thermolysis process for the production of compound (15).

¹H-NMR (Benzene-d₆, 200 MHz) δ=6.26 (s, 1H), 5.23 (s, 1H), 3.84-3.74 (q, 2H), 0.82 (t, 3H).

What is claimed is:

1. A process for the synthesis of 1,1-disubstituted olefins, including
in a first step the reaction of a 1,3-diene with an olefin of the general Formula (I), 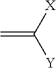
in which X is an electron withdrawing substituent and Y is a hydrogen atom or a halide atom, wherein X and Y are not identical,
to an unsaturated cyclic compound comprising the substituents X and Y;
in a second step the substitution of Y by a substituent W, selected from a C₁-C₁₂ hydrocarbon group, a C₂-C₁₂ carboxylic acid ester group, a C₃-C₁₂ alkoxy carbonylalkyl group, a C₂-C₁₂ alkanoyl, C₄-C₁₂ cycloalkanecarbonyl or C₆-C₁₂ (hetero)arenecarbonyl group, a C₁-C₁₂ hydroxyalkyl group, a C₂-C₁₂ phosphonic acid ester group, a C₁-C₁₂ sulfonyl group, a C₁-C₁₂ sulfinyl group, a C₁-C₁₂ sulfonic acid ester group, an aldehyde group, a carbamoyl group, a halide atom and a cyano group; and
in a third step the elimination of a 1,1-disubstituted olefin of the general Formula 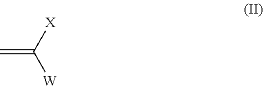
in which X and W have the above meaning.

2. The process according to claim 1, wherein X comprises at least one carbon atom and at least one heteroatom, selected from nitrogen, sulfur and oxygen.

3. The process according to claim 1, wherein X is a cyano (—CN) group or a carboxylic acid alkyl ester group —COOR, in which R is an alkyl group.

4. The process according to claim 1, wherein the unsaturated, X,Y-substituted cyclic compound obtained in the first step is a bicyclic or polycyclic compound.

5. The process according to claim 1, wherein the stoichiometric ratio of the olefin of the general Formula (I) to the 1,3-diene in the first step is at least 3:1.

6. The process according to claim 1, wherein Y is a hydrogen atom.

7. The process according to claim 6, wherein the substitution of Y by W includes at least two partial steps:
a first partial step, in which a reactive species is formed from the reaction of a base with the unsaturated, X,Y-substituted, cyclic compound;
a second partial step, in which a compound capable of contributing the substituent W is added to the reactive species.

8. The process according to claim 6, wherein the substitution of Y by a substituent W is carried out at temperatures below 0° C.

9. The process according to claim 1, wherein X is a cyano group and W is a carboxylic acid ester group.

10. The process according to claim 1, wherein the stoichiometric ratio of the compound capable of contributing the substituent W to the unsaturated, X,Y-substituted, cyclic compound has a value between 1:1 to 3:1 in the second step.

11. The process according to claim 1, wherein the elimination of the 1,1-disubstituted olefin of the general Formula (II) is effected thermally or by electromagnetic induction.

12. The process according to claim 11, wherein the elimination of the 1,1-disubstituted olefin of the general Formula (II) is effected by electromagnetic induction, in that an unsaturated, X,Y-substituted, cyclic compound is brought into contact with a solid heating medium that can be heated by electromagnetic induction, said medium being inside a reactor and is heated by electromagnetic induction with the aid of an inductor, wherein the 1,1-disubstituted olefin of the general Formula (II) is formed from the unsaturated, X,Y-substituted cyclic compound.
